(19) United States
(12) Patent Application Publication
(10) Pub. No.: US 2007/0213354 A1
(43) Pub. Date: Sep. 13, 2007

(54) Compositions and Methods for the Treatment of Multiple Sclerosis

(75) Inventors: Robert T. Dorr, Tucson, AZ (US); Evan M. Hersh, Tucson, AZ (US); David S. Alberts, Tucson, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AmpliMed Corporation, Tucson, AZ (US)

(21) Appl. No.: 11/681,120

(22) Filed: Mar. 1, 2007

Related U.S. Application Data
(60) Provisional application No. 60/780,710, filed on Mar. 8, 2006.

(51) Int. Cl. A61K 31/473

(52) U.S. Cl. 514/288

(57) ABSTRACT

Compositions and methods for treating multiple sclerosis are disclosed herein. Embodiments of the present invention include methods of treating a multiple sclerosis patient via the administration of a therapeutically effective amount of a composition comprising a compound of the formula: 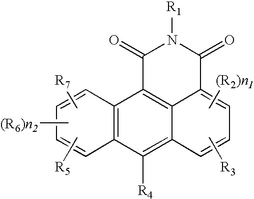 wherein,
R₁ is heteroalkyl, heterocycloalkyl, or heteroaryl;
R₂, R₄ and R₆ are independently hydrogen, halogen, nitro, amino, hydroxy, C₁-C₆ alkyl, C₁-C₆ heteroalkyl, heterocycloalkyl or aryl;
R₃, R₅, and R₇ are independently hydrogen or C₁-C₆ alkyl, or
R₃ and R₄ taken together with the carbon atoms to which they are attached form a phenyl ring, or
R₄ and R₅ taken together with the carbon atoms to which they are attached form a phenyl ring, or
R₅ and R₇ taken together with the carbon atoms to which they are attached form a phenyl ring; and
n₁ and n₂ are independently 0, 1 or 2; or a pharmaceutically acceptable salt, a prodrug, or a mixture thereof.

### CROSS REFERENCE TO RELATED APPLICATIONS

This application claims priority to U.S. provisional patent application No. 60/780,710, filed Mar. 8, 2006, the disclosure of which is hereby incorporated by reference in its entirety for all purposes.

### FIELD OF THE INVENTION

This disclosure relates generally to pharmaceutical compositions and methods for the treatment of multiple sclerosis, and more particularly, but not exclusively, to methods of preventing disability caused by disease progression, to methods of reducing the frequency, severity, or duration of a disease relapse, and to methods of relieving one or more symptoms of multiple sclerosis.

### BACKGROUND INFORMATION

Multiple sclerosis (“MS”) is a chronic disorder of the central nervous system (“CNS”) characterized by an inflammatory phase and a neurodegenerative phase, leading to demyelination, axon loss, and atrophy of portions of the CNS, resulting in disability and ultimately death.

The disease assumes several different clinical patterns in patients, the most common form being relapsing-remitting MS. Relapsing-remitting MS is characterized by clearly defined disease relapses with either full recovery or with sequelae and residual deficiencies upon recovery. Most patients with relapsing-remitting MS ultimately develop secondary progressive MS, which is characterized by disease progression with or without occasional relapses, minor remissions, or plateaus. Another less common clinical pattern, occurring in approximately 10% of new MS patients, is primary progressive MS, which is characterized by disease progression from the onset, with only occasional plateaus and temporary improvements in disease symptoms. The least common form of MS is progressive-relapsing MS, characterized by disease progression from the outset with acute relapses, with or without full recovery, and wherein the periods between relapses are characterized by continuous disease progression. Rizvi, S. A. et al., Neurology 2004; 63 (Suppl 6): S8-S14.

While the etiology of MS, in any of its observed clinical patterns, remains unknown, several lines of evidence support the hypothesis that autoimmunity plays a major role in the development of the disease. Research suggests the presence of auto-antibodies recognizing myelin antigens in MS patients such as myelin basic protein, myelin oligodendrocyte glycoprotein, proteolipid protein, myelin-associated glycoprotein, and other CNS antigens. Correale J. et al., J. of Neuroimmunology 162 (2005) 173-183.

Existing treatment options specifically target the inflammatory phase of MS and include immunomodulators interferon β and glatiramer acetate, and an immunosuppressant, mitoxantrone. While the interferon βs and glatiramer acetate have demonstrated effectiveness in delaying the onset of the progressive stage of the disease, only mitoxantrone has been approved by the U.S. Food and Drug Administration for the treatment of the progressive disease patterns described previously. Rizvi, S. A. et al., supra.

Mitoxantrone is an anthracenedione with anti-inflammatory and immunomodulating properties, and suppresses both B and T lymphocytes. Effects on B cells lead to a decrease in the rate and magnitude of B-cell function, thereby decreasing antibody formation. Jeffrey, D. R. et al., Neurology 2004; 63 (Suppl 6): S19-S24. Unfortunately, a major limitation to the use of mitoxantrone in the treatment of MS is its potential to produce cardiotoxicity, which restricts the cumulative lifetime dose of the drug to 140 mg/m² of body surface area. Rizvi, S. A. et al., supra.

### SUMMARY OF THE INVENTION

The present invention is directed to pharmaceutical compositions comprising 1,2-dihydro-3H-dibenz(deh)isoquinoline-1,3-dione derivatives which are useful in the treatment of MS and to methods for the treatment of MS. In one aspect, the present invention provides a method of treating a patient exhibiting a symptom of MS, wherein the method comprises administering a therapeutically effective amount of a pharmaceutical composition comprising a compound of the formula: 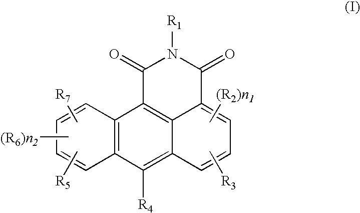
wherein,
R₁ is heteroalkyl, heterocycloalkyl, or heteroaryl;
R₂, R₄ and R₆ are independently hydrogen, halogen, nitro, amino, hydroxy, C₁-C₆ alkyl, C₁-C₆ heteroalkyl, heterocycloalkyl or aryl;
R₃, R₅, and R₇ are independently hydrogen or C₁-C₆ alkyl, or
R₃ and R₄ taken together with the carbon atoms to which they are attached form a phenyl ring, or
R₄ and R₅ taken together with the carbon atoms to which they are attached form a phenyl ring, or
R₅ and R₇ taken together with the carbon atoms to which they are attached form a phenyl ring; and
n₁ and n₂ are independently 0, 1 or 2;
or a pharmaceutically acceptable salt, a prodrug, or a mixture thereof.

In another aspect, the present invention is directed to a method of preventing disease progression and/or disability caused by disease progression via the administration of a therapeutically effective amount of a composition comprising a compound of Formula (I) with substituents as defined hereinbefore, or a pharmaceutically acceptable salt, a prodrug, or a mixture thereof.

In yet another aspect, the present invention provides a method of reducing the frequency, severity, or duration of a relapse via the administration of a therapeutically effective amount of a composition comprising a compound of Formula (I) with substituents as defined hereinbefore, or a pharmaceutically acceptable salt, a prodrug, or a mixture thereof.

In still another aspect, the present invention is directed to a method of relieving, or preventing the progression of, one or more clinical symptoms of MS via the administration of a therapeutically effective amount of a composition comprising a compound of Formula (I) with substituents as defined hereinbefore, or a pharmaceutically acceptable salt, a prodrug, or a mixture thereof.

### BRIEF DESCRIPTION OF THE DRAWINGS

FIG. 1 is a graphical illustration of the effects of 0.125 mg/kg ethonafide (AMP) administration to mice beginning on the date of immunization for the acute monophasic form of EAE in comparison to untreated and mitoxantrone-treated controls.

FIG. 2 is a graphical illustration of the effects of 0.25 mg/kg ethonafide (AMP) administration to mice beginning on the date of immunization for the acute monophasic form of EAE in comparison to untreated and mitoxantrone-treated controls.

FIG. 3 is a graphical illustration of the effects of 0.5 mg/kg ethonafide (AMP) administration to mice beginning on the date of immunization for the acute monophasic form of EAE in comparison to untreated and mitoxantrone-treated controls.

FIG. 4 is a graphical illustration of the effects of 0.5 mg/kg and 1.0 mg/kg ethonafide (AMP) administration to mice beginning on day 11 following immunization for the acute monophasic form of EAE in comparison to untreated and mitoxantrone-treated controls.

### DETAILED DESCRIPTION OF EMBODIMENTS OF THE INVENTION

Embodiments of pharmaceutical compositions and methods for the treatment of multiple sclerosis are disclosed herein. In the following description, numerous specific details are provided, such as the identification of various components and structures, to provide a thorough understanding of embodiments of the invention. One skilled in the art will recognize however, that the invention can be practiced without one or more of the specific details, or with other methods, components, materials, and the like. In still other instances, well-known components, materials, or processes are not shown or described in detail to avoid obscuring aspects of various embodiments of the invention.

Reference throughout this specification to “one embodiment” or “an embodiment” means that a particular feature, structure, component, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, components, or characteristics may be combined in any suitable manner in one or more embodiments.

As described hereinbefore, the present invention is directed to compositions and methods for treating MS via the administration of pharmaceutical compositions comprising 1,2-dihydro-3H-dibenz(deh)isoquinoline-1,3-dione derivatives. Because the dibenzisoquinoline ring structure may have substituents at various positions, to aid in the understanding of the various derivatives to which the invention pertains, the nomenclature with respect to the dibenzisoquinoline structure is as follows:

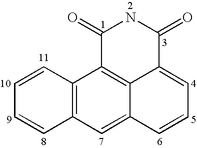

### I. Definitions

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “therapeutically effective amount” refers to the amount of a compound or composition effective to yield the desired therapeutic response. The therapeutically effective amount may vary with such factors as the particular condition or clinical pattern of disease being treated, the physical condition of the patient, the duration of the treatment, the nature of concurrent therapy (if any), and the specific formulations employed.

The term “alkyl,” by itself or as part of another substituent, means, unless otherwise stated, a straight (i.e. unbranched) or branched chain hydrocarbon radical which may be fully saturated, mono- or polyunsaturated and can include di- and multivalent radicals, having the number of carbon atoms designated (e.g. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Similarly, the term “alkylene” by itself or as part of another substituent means a divalent radical derived from alkyl, as exemplified, but not limited by, methylene (—CH₂—), ethylene (—CH₂—CH₂—), propylene (—CH₂—CH₂—CH₂—), and isopropylene (—CH₂(CH₃)—CH₂—).

The term “heteroalkyl,” by itself or in combination with another term, means, unless otherwise stated, a stable straight or branched chain hydrocarbon radical consisting of at least one carbon atom and at least one heteroatom selected from the group consisting of O, N and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the heteroalkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, —CH═CH—N(CH₃)—CH₃, —O—CH₃, —O—CH₂—CH₃, —NH—CH₂—OH, —CH(OH)—CH₃, —C(O)—CH₂—OH, —C(O)—CH₂—O—C(O)—CH₂—CH₃, —O—C(O)—C(CH₃)₃, and —O—C(O)—CH₂—CH₃. Up to three heteroatoms may be consecutive, such as, for example, —CH₂—NH—OCH₃ and —N═N—N(CH₃)₂. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopropylmethyl, cyclopentyl, cyclohexyl, cyclohexylmethyl, 1-cyclohexenyl, 3-cyclohexenyl, cycloheptyl, and the like. Examples of heterocycloalkyl include, but are not limited to, 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl 1-pyrrolidinyl, 2-pyrrolidinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent derivatives of aryl and heteroaryl, respectively.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein, is meant to cover only aryls substituted with one or more halogens.

The term “oxo” means an oxygen that is double bonded to a carbon atom.

Each of above terms (e.g., “alkyl,” “heteroalkyl,” “cycloalkyl,” “heterocycloalkyl,” “aryl,” “heteroaryl,” and “arylalkyl,” as well as their divalent radical derivatives) are meant to include both substituted and unsubstituted forms of the indicated radical. Preferred substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″ and R′″ each preferably independently refer to hydrogen, or C₁-C₆ alkyl, cycloalkyl, or haloalkyl. Unless otherwise stated, when a compound of the invention includes more than one R group, each of the R groups is independently selected as are each R′, R″ and R′″ groups when more than one of these groups is present.

Similar to the substituents described for alkyl radicals above, exemplary substituents for aryl and heteroaryl groups (as well as their divalent derivatives) are varied and are selected from, for example: —OR′, —NR′R″, —SR′, -halogen, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on the aromatic ring system; and where R′, R″, R′″ and R″″ are preferably independently selected from hydrogen, or C₁-C₆ alkyl, cycloalkyl, or haloalkyl. Unless otherwise stated, when a compound of the invention includes more than one R group, for example, each of the R groups is independently selected as are each R′, R″, R′″ and R″″ groups when more than one of these groups is present.

Two of the substituents on adjacent atoms of an aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of an aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of an aryl or heteroaryl ring may optionally be replaced with a substituent of the formula —(CRR″)ₛ—X′—(CR″R′″)_{d}—, where s and d are independently integers of from 0 to 3, and X′ is —O—, —NR″—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), and sulfur (S).

The compounds of the present invention may exist as salts. The present invention includes such salts. These salts may be prepared by methods known to those skilled in art. The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lacetic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

### II. 1,2-dihydro-3H-dibenz(deh)isoquinoline-1,3-diones of the Present Invention

Compounds useful in accordance with embodiments of the present invention comprise 1,2-dihydro-3H-dibenz(deh)isoquinoline-1,3-diones of the formula: 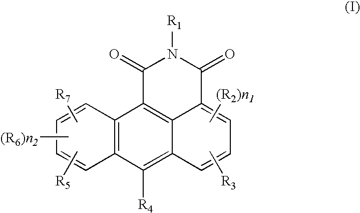
wherein,
R₁ is heteroalkyl, heterocycloalkyl, or heteroaryl;
R₂, R₄ and R₆ are independently hydrogen, halogen, nitro, amino, hydroxy, C₁-C₆ alkyl, C₁-C₆ heteroalkyl, heterocycloalkyl or aryl;
R₃, R₅, and R₇ are independently hydrogen or C₁-C₆ alkyl, or
R₃ and R₄ taken together with the carbon atoms to which they are attached form a phenyl ring, or
R₄ and R₅ taken together with the carbon atoms to which they are attached form a phenyl ring, or

R₅ and R₇ taken together with the carbon atoms to which they are attached form a phenyl ring; and

n₁ and n₂ are independently 0, 1 or 2;

or a pharmaceutically acceptable salt, a prodrug, or a mixture thereof.

1,2-dihydro-3H-dibenz(deh)isoquinoline-1,3-diones in accordance with preferred embodiments of the present invention include compounds of the formula: 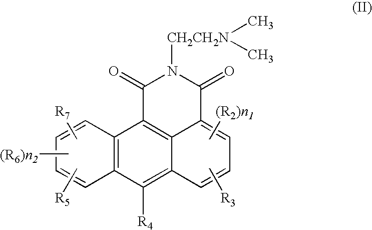
wherein,
R₂, R₃, R₄, R₅, R₆, and R₇ are independently selected from hydrogen, halogen, nitro, amino, hydroxy, C₁-C₆ alkyl, and C₁-C₆ heteroalkyl; and
n₁ and n₂ are independently 0, 1 or 2. Compounds of Formula (II) may be referred to herein as azonafide compounds.

In a particularly preferred embodiment, 1,2-dihydro-3H-dibenz(deh)isoquinoline-1,3-diones of the present invention include compounds of Formula (II) wherein R₃, R₄, R₅, R₆, and R₇ are hydrogen, and R₂ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃. In one embodiment, n₁ is 1.

In another particularly preferred embodiment, 1,2-dihydro-3H-dibenz(deh)isoquinoline-1,3-diones of the present invention include compounds of Formula (II) wherein R₂, R₃, R₅, R₆, and R₇ are hydrogen, and R₄ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

In yet another particularly preferred embodiment, 1,2-dihydro-3H-dibenz(deh)isoquinoline-1,3-diones of the present invention include compounds of Formula (II) wherein R₂, R₃, R₄, R₅, and R₇ are hydrogen, and R₆ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃. In one embodiment, n₂ is 1.

### A. Methods for Preparing 1,2-dihydro-3H-dibenz(deh)isoquinoline-1,3-diones

Compounds useful in accordance with embodiments of the present invention can be prepared by art-recognized techniques. More specifically, the dibenzisoquinoline nucleas can be prepared via the following exemplary procedure:

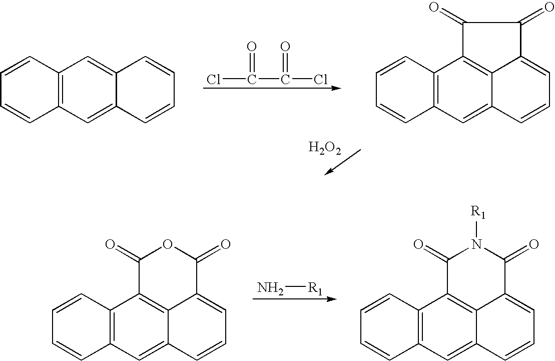

The addition or modification of substituents at positions 4-11 of the dibenzisoquinoline ring structure may be accomplished via the synthetic schema described and illustrated in the following references: Sami et al., J Med. Chem. 38: 983-993 (1995); Sami et al., J Med. Chem. 39: 1609-1618 (1996); Sami et al., J Med. Chem. 39: 4978-4987 (1996); and U.S. Pat. No. 5,635,506, issued Jun. 3, 1997 to Alberts et al., each expressly incorporated herein by reference in its entirety.

### B. Assays for Testing the Therapeutic Activity of 1,2-dihydro-3H-dibenz(deh)isoquinoline-1,3-diones

Compounds useful in accordance with embodiments of the present invention were tested for their in vitro activity in both sensitive and resistant cell lines to evaluate their ability to suppress immune system cells. The efficient suppression of immune system cells is thought to be a desirable characteristic in therapies intended to treat, delay the onset of, or diminish, the symptoms of MS and the disability resulting therefrom.

Murine leukemia cell lines (L1210) having a sensitivity or resistance to standard cytotoxic agents were utilized to evaluate the immunosuppressive effects of compounds in accordance with the present invention. The resistant cell line produces the membrane protein P-glycoprotein, which acts as a drug efflux pump, expelling a wide variety of standard cytotoxic agents such as doxorubicin, vinca alkoloid compounds, and other DNA binding agents. The murine leukemia experiments were based on continuous drug exposure using the MTT assay as described in Mosmann, T., J Immunol Met. 65: 55-63 (1983) and Alley et al., Cancer Res. 48: 589-601 (1988), whereby cell viability is measured by the reduction of the tetrazolium salt MTT to a colored formazan salt, which can then be quantitated by a calorimetric assay. The results of the MTT assays are shown in Table 1 hereinafter.

TABLE 1

| | | | IC₅₀ (nM) | | |
|---|---|---|---|---|---|
| L1210 | L1210 | | Cardiotox: | | |
| sensitive | resistant | Cardiotox^{†} | L1210sens^{††} | Compound | Reference |
| 7 | 7 | 1983 | 283.29 | 2-(CH₂)₂N(CH₃)₂ | 1 |
| 10 | 21 | 7060 | 706.00 | 2-(CH₂)₂NHCH₃ | 1 |
| 54 | 82 | 11410 | 211.30 | 2-(CH₂)₃N(CH₃)₂ | 1 |
| 18 | 41 | 943 | 52.39 | 2-(CH₂)₂NH(CH₂)₂OH | 1 |
| 76 | 142 | 8910 | 117.24 | 2-(CH₂)₃N(CH₂CH₂OH)₂ | 1 |
| 15 | 12 | 920 | 61.33 | 2-(CH₂)₂N(CH₂)₄ | 1 |
| 51 | 51 | 10400 | 203.92 | 2-(CH₂)₂N(CH₂)₅ | 1 |
| 51 | 56 | 7080 | 138.82 | 2-(CH₂)₂-(1-methyl-2-pyrrolidinyl) | 1 |
| 36 | 55 | 15200 | 422.22 | 2-CH₂-(1-ethyl-2-pyrrolidinyl) | 1 |
| 635 | 609 | 8760 | 13.80 | 2-(1-ethyl-3-piperidinyl) | 1 |
| 512 | 512 | >25000 | 48.83 | 2-(CH₂)₂—N-morpholinyl | 1 |
| 174 | 463 | 6713 | 38.58 | 2-(CH₂)₂—N-piperazinyl | 1 |
| 6443 | 15464 | >25000 | 3.88 | 2-(CH₂)₂-2-pyridyl | 1 |
| 26738 | 26740 | >25000 | 0.93 | 2-CH₂-2-pyridyl | 1 |
| 1872 | 2674 | >25000 | 13.35 | 2-CH₂-3-pyridyl | 1 |
| 544 | 554 | >25000 | 45.96 | 2-(3-pyridyl) | 1 |
| 187 | 1791 | 37313 | 199.53 | 2-(p-N(CH₃)₂)—C₆H₅) | 1 |
| 54 | 68 | 1357 | 25.13 | *4-CH₃ | 2 |
| 10 | 15 | 900 | 90.00 | *4-Cl | 2 |
| 5 | 8 | 434 | 86.80 | *4-NH₂ | 4 |
| 608 | 560 | >>24331 | 40.02 | *4-NHCOCH₃ | 4 |
| 862 | 172 | >24630 | 28.57 | *4-NHCOC(CH₃)₃ | 4 |
| 210 | 245 | 734 | 3.50 | *4-NH(CH₂)₂N(CH₃)₂ | 2 |
| 6 | 15 | 1724 | 287.33 | *5-NH₂ | 4 |
| 19 | 73 | 177 | 9.32 | *5-NHCOCH₃ | 4 |
| 44 | 15 | 662 | 15.05 | *5-NHCOC(CH₃)₃ | 4 |
| 7 | 27 | 989 | 141.29 | *6-CH₃ | 3 |
| 183 | 235 | 7833 | 42.80 | *6-C₂H₅ | 3 |
| 77 | 64 | 4113 | 53.42 | *6-Cl | 3 |
| 416 | 416 | 43704 | 105.06 | *6-I | 3 |
| 217 | 145 | >24096 | 111.04 | *6-OH | 3 |
| 3 | 4 | 799 | 266.33 | *6-OCH₃ | 3 |
| 8 | 7 | 1779 | 222.38 | *6-OC₂H₅ | 3 |
| 17 | 49 | 7758 | 456.35 | *6-OC₃H₇ | 3 |
| 0.2 | 1 | 5439 | 27195.00 | *6-O(CH₂)₂N(CH₃)₂ | 3 |
| 0.4 | 0.5 | 3000 | 7500.00 | *6-SCH₃ | 3 |
| 6 | 5 | 7738 | 1289.67 | *6-SC₂H₅ | 3 |
| 35 | 35 | 2315 | 66.14 | *6-SO₂CH₃ | 3 |
| 5 | 27 | 1486 | 297.20 | *6-NH₂ | 3 |
| 486 | 729 | 12880 | 26.50 | *6-NHCOCH₃ | 3 |
| 23 | 96 | 46599 | 2026.04 | *6-N(CH₃)₂ | 3 |
| 6 | 48 | 1209 | 201.50 | *6-NH(CH₂)₂OH | 3 |
| 0.04 | 0.04 | 4193 | 104825.00 | *6-NH(CH₂)₂N(CH₃)₂ | 3 |
| 34 | 70 | 1685 | 49.56 | *7-CH₃ | 3 |
| 154 | 206 | 10797 | 70.11 | *7-Cl | 3 |
| 472 | 212 | >24096 | 51.05 | *7-OH | 3 |
| 117 | 109 | 26042 | 222.58 | *7-OCH₃ | 3 |
| 63 | 50 | 5300 | 84.13 | *7-OC₂H₅ | 3 |
| 6250 | 1225 | >50000 | 8.00 | *7-SCH₃ | 3 |
| 5 | 7 | 1951 | 390.20 | *7-NH₂ | 3 |
| 487 | 487 | >23613 | 48.49 | *7-NHCOCH₃ | 3 |
| 524 | 524 | 290634 | 554.65 | *7-NH(CH₂)₂N(CH₃)₂ | 3 |
| 108 | 67 | 2949 | 27.31 | *8-OH | 2 |
| 13 | 18 | 806 | 62.00 | *8-OCH₃ | 2 |
| 6775 | 6775 | >81300 | 12.00 | *8-NH₂ | 4 |
| 485 | 510 | 12136 | 25.02 | *8-NHCOCH₃ | 4 |
| 50 | 20 | 650 | 13.00 | *8-NO₂ | 4 |
| 8 | 8 | 1542 | 192.75 | *9-Cl | 2 |
| 5 | 7 | 404 | 80.80 | *9-OH | 2 |
| 54 | 54 | <2597 | 48.09 | *9-OCH₃ | 2 |
| 3 | 16 | 813 | 271.00 | *9-NH₂ | 4 |
| 114 | 170 | 487 | 4.27 | *9-NHCOCH₃ | 4 |
| 41 | 54 | 1004 | 24.49 | *10-CH₃ | 2 |
| 15 | 13 | 105 | 7.00 | *10-CN | 2 |
| 8 | | 456 | 57.00 | *10-F | 2 |
| 5 | 8 | 771 | 154.20 | *10-Cl | 2 |
| 312 | 52 | 4787 | 15.34 | *10-I | 2 |
| 189 | 540 | >26990 | 142.80 | *10-OH | 2 |
| 21 | 52 | 9103 | 433.48 | *10-OCH₃ | 2 |
| 25 | 50 | 11292 | 451.68 | *10-OC₂H₅ | 2 |
| 3 | 3 | 943 | 314.33 | *10-NH₂ | 4 |
| 49 | 219 | 7786 | 158.90 | *10-NHCOCH₃ | 4 |
| 200 | 200 | 11025 | 55.13 | *10-NHCOC(CH₃)₃ | 4 |
| 25 | 25 | 801 | 32.04 | *10-NO₂ | 2 |
| 1779 | 617 | 8226 | 4.62 | *11-Cl | 2 |
| 189 | 189 | 15424 | 81.61 | *11-OH | 2 |
| 678 | 678 | 27100 | 39.97 | *11-NH₂ | 4 |
| 243 | 2433 | >22500 | 92.59 | *11-NHCOCH₃ | 4 |
| 23 | 50 | 8647 | 375.96 | *11-NO₂ | 4 |
| 53 | 53 | 7600 | 143.40 | 2-(CH₂)₂NHCH₃; 6-Cl | 3 |
| 41 | 27 | 8097 | 197.49 | 2-(CH₂)₂NHCH₃; 6-OCH₃ | 3 |
| 3 | 7 | 3226 | 1075.33 | *6-NH(CH₂)₂N(CH₃)₂; 8-Cl | 3 |
| 10 | 39 | 7737 | 773.70 | Mitoxantrone | 2 |
| 35 | 3884 | 10151 | 290.03 | Doxorubicin | 2 |

*includes 2-(CH₂)₂N(CH₃)₂
Reference 1: Sami et al., J Med Chem. 36: 765-770 (1993)
Reference 2: Sami et al., J Med Chem. 39: 4978-4987 (1996)
Reference 3: Sami et al., J Med Chem. 39: 1609-1618 (1996)
Reference 4: Sami et al., J Med Chem. 38: 983-993 (1995)
^{†}IC₅₀ is at 1 hour, and corresponds to the drug concentration that reduces the ATP/protein ratio to 50% of that in untreated control myocytes.
^{††}Ratio of the IC₅₀ values reported for Cardiotox and L1210 sensitive assays.

Because cardiotoxicity limits the effectiveness of other anthracene derivatives, including mitoxantrone, the relative cardiotoxicity of the compounds useful in accordance with the present invention was also investigated. Cardiotoxicity was determined by a neonatal rat myocyte assay, as described in Dorr et al., Cancer Res. 48: 5222-5227 (1988), wherein cardiotoxicity is measured by the ATP/protein ratio compared with untreated controls. The IC₅₀ is the 1 hour drug concentration that reduces this ratio to 50% of that in untreated control myocytes. The results of the cardiotoxicity assays are shown in Table 1.

In order to compare the relative toxicity and immune suppressive characteristics of the compounds useful in accordance with the present invention, a ratio of the cardiotoxicity to the activity in the L1210 sensitive cell line (“cardiotox ratio”) was calculated, and is shown in Table 1. As can be seen by a review of the data, several of the compounds useful in accordance with the present invention show not only a much greater effectiveness in suppressing immune system cells, as compared to mitoxantrone, but also a much reduced relative cardiotoxicity as compared to mitoxantrone or another antracene derivative, doxorubicin. In particular, compounds substituted at the 6 position of the dibenzisoquinoline ring structure with —O(CH₂)₂N(CH₃)₂, —SCH₃, —SCH₂CH₃, —N(CH₃)₂, and —NH(CH₂)₂N(CH₃)₂, as well as a compound substituted at the 6 position with —NH(CH₂)₂N(CH₃)₂ and also at the 8 position with —Cl, showed much greater cardiotox ratios than did the mitoxantrone control.

The results of the murine leukemia investigations demonstrate that compositions comprising compounds of Formula (I) or (II), wherein R₁, R₂, R₃, R₄, R₅, R₆, and R₇ are as described hereinbefore, are likely to be useful in methods of treating MS patients when administered in therapeutically effective amounts. Moreover, compositions comprising compounds of Formula (I) or (II), wherein R₁, R₂, R₃, R₄, R₅, R₆, and R₇ are as described hereinbefore, are likely to be useful in methods of preventing progression to symptomatic disease, methods of preventing disability caused by disease progression, methods of reducing the frequency, severity, or duration of a disease relapse, and in methods of relieving one or more symptoms of MS when administered in therapeutically effective amounts.

The principal preclinical model for confirming the efficacy of therapeutic compounds in the treatment or prevention of symptomatic MS is the murine experimental autoimmune encephalomyelitis (“EAE”) model. The EAE model is a well-studied system that enables the induction of varying forms of the disease in mice to mimic the several clinical patterns of MS seen in humans, and thereby provides a mechanism to ascertain the efficacy of potential treatments for MS in human patients. The varying forms of EAE can be induced in mice via the administration of peptides derived from myelin proteins, i.e., proteolipid protein₁₃₉₋₁₅₁ (“PLP”), myelin oligodendrocyte glycoprotein₃₅₋₅₅ (“MOG”), or myelin basic protein₈₅₋₉₉ (“MBP”).

An acute monophasic form of EAE, induced by the administration of MOG, mimics the progressive clinical patterns of MS seen in human patients and can provide an opportunity to investigate a therapy's ability to prevent progression to symptomatic disease, to prevent or reduce the disability that would otherwise be caused by disease progression, or to treat one or more clinical symptoms of MS. The induction of the acute monophasic form of EAE in C57/BL6 mice can be achieved as described in Gaupp et al., Am J Pathol. 162(1): 139-150 (2003). Similarly, a relapsing-remitting form of EAE, induced by the administration of PLP, mimics the more common relapsing-remitting form of MS seen in humans and can provide an opportunity to investigate potential therapies to reduce the frequency, severity, or duration of a disease relapse, to otherwise prevent disability caused by disease progression, or to relieve one or more clinical symptoms of MS in human patients. The induction of the relapsing-remitting form of EAE in SJL/J mice can be achieved as described in Fridkis-Hareli et al., J Clin Invest. 109(12): 1635-1643 (2002).

Acute monophasic EAE was induced in C57/BL6 mice by subcutaneous flank and tail base injections of 200 μg of MOG in complete Freund's adjuvent containing 500 μg of heat-inactivated mycobacterium tuberculosis on day 0, supplemented by intravenous injections of 200 ng of pertussis toxin on day 2.

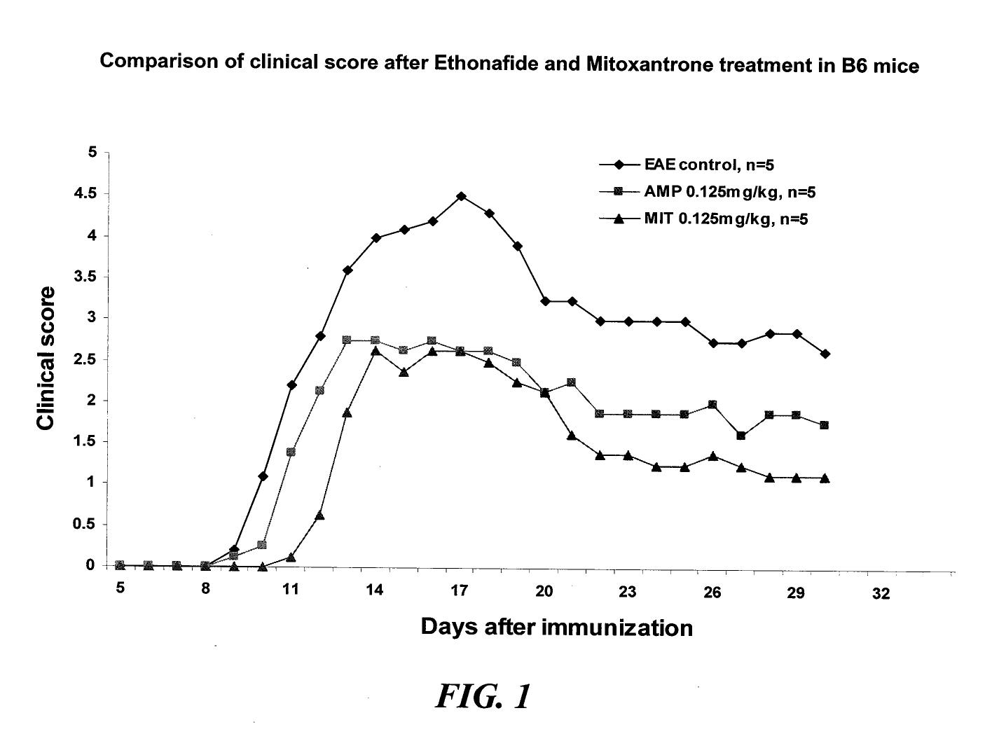

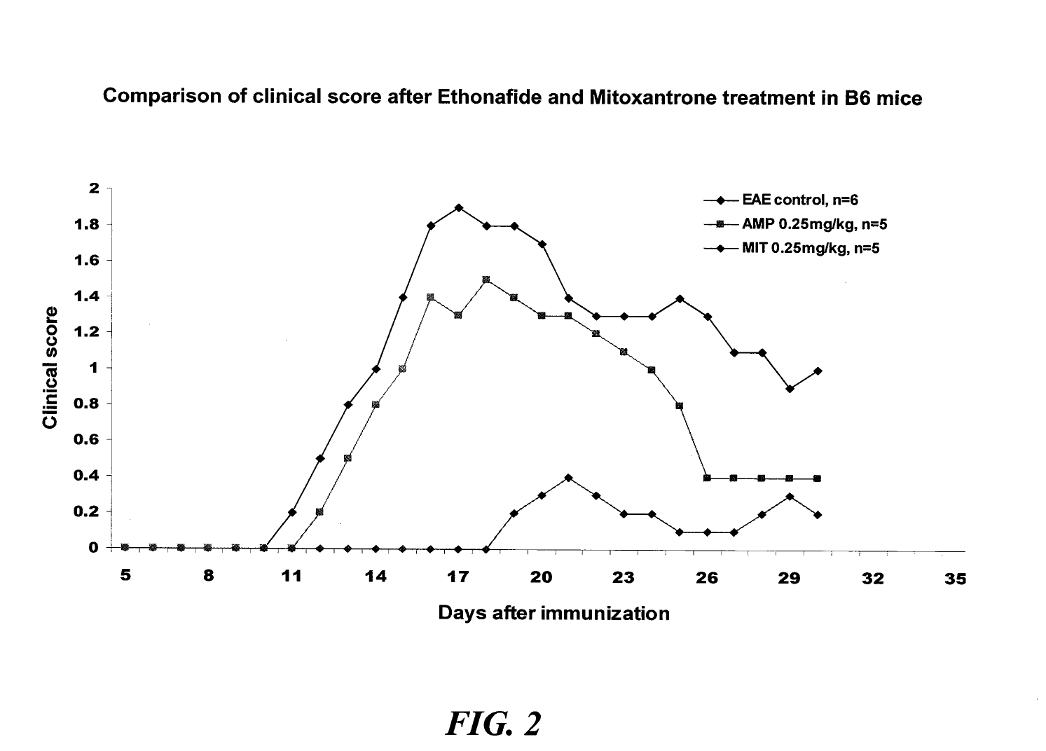

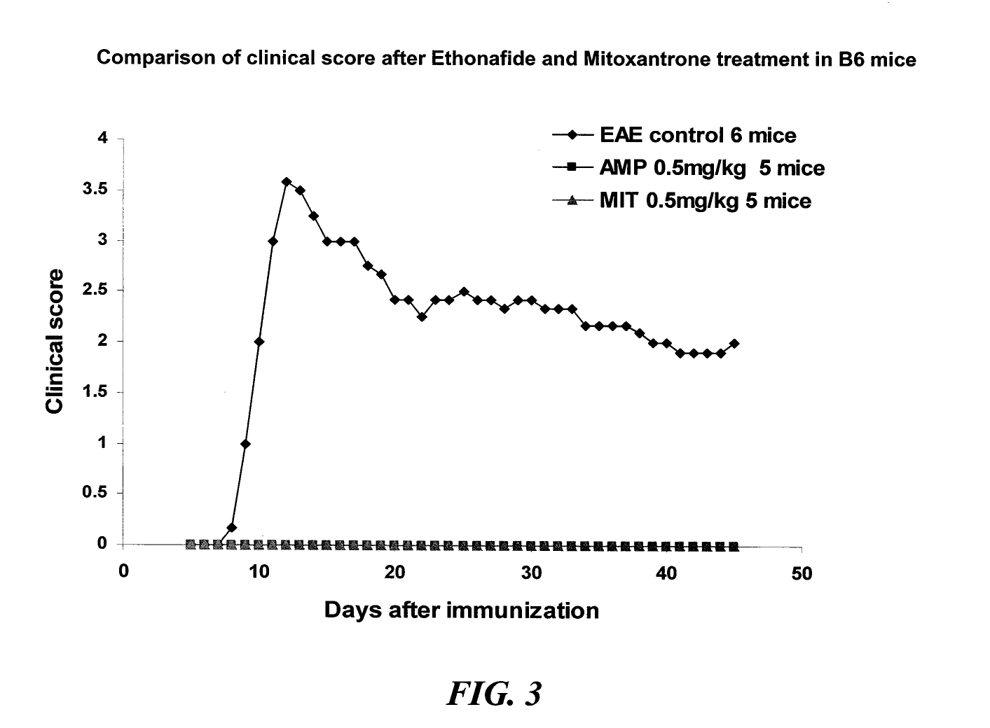

With reference first to FIGS. 1-3, beginning on the day of immunization for acute monophasic EAE, nine groups of mice (each group containing 5 or 6 individuals) were treated with either ethonafide (AMP; a compound of Formula (II) wherein the dibenzisoquinoline ring structure is substituted at position 6 with —OCH₂CH₃) or mitoxantrone at a dose of 0.125 mg/kg, 0.25 mg/kg, or 0.5 mg/kg daily for seven days, or were left untreated to be used as a control at each respective dose level. Each drug was administered intraperitoneally in phosphate buffered saline (untreated mice received i.p. administrations of the PBS vehicle). The mice were observed daily for clinical signs of disease, and were scored (by multiple investigators) on an arbitrary scale from 0 to 5 in increments of 0.5 according to the following parameters: 0—no clinical signs of disease; 1—flaccid tail; 2—hind limb weakness or abnormal gait; 3—complete hind paralysis; 4—complete hind paralysis with forelimb weakness or paralysis; and 5-moribund or deceased. The incidence of disease in the immunized mice, the day of disease onset, the maximum clinical score, and the mean clinical score over a 30 day observation period were determined, and the results are shown in Table 2.

TABLE 2
Acute Monophasic EAE - Drug Administration beginning with Immunization

| Drug - Dose (mg/kg) | Incidence | | Day of Onset | Max. Clinical Score | Mean Clinical Score* |
|---|---|---|---|---|---|
| PBS Vehicle | 100% | (5/5) | 10.2 ± 0.38 | 4.6 ± 0.19 | 3.06 ± 0.68 |
| Ethonafide - 0.125 | 100% | (5/5) | 11.5 ± 0.88 | 3.5 ± 0.22 | 1.98 ± 0.48 |
| Mitoxantrone - 0.125 | 100% | (5/5) | 12.5 ± 0.67 | 3.1 ± 0.33 | 1.63 ± 0.59 |
| PBS Vehicle | 100% | (6/6) | 14.3 ± 1.3 | 2.5 ± 0.5 | 1.25 ± 0.34 |
| Ethonafide - 0.25 | 100% | (5/5) | 17.8 ± 3.4 | 1.8 ± 1.16 | 0.88 ± 0.38 |
| Mitoxantrone - 0.25 | 40% | (2/5) | 19, 28 | 0.4, 0.3 | 0.21 ± 0.07 |
| PBS Vehicle | 100% | (6/6) | 9.0 ± 1.3 | 4.0 ± 0.5 | 2.2 ± 0.37 |
| Ethonafide - 0.5 | 0% | (0/5) | NA | 0 | 0 |
| Mitoxantrone - 0.5 | 0% | (0/5) | NA | 0 | 0 |

*calculated for mice that had evidence of disease.

As illustrated in FIGS. 1-3, and in Table 2, the administration of ethonafide demonstrated a dose dependent effect in delaying or preventing the onset of clinically symptomatic progressive disease and the disability or severity of disability caused thereby.

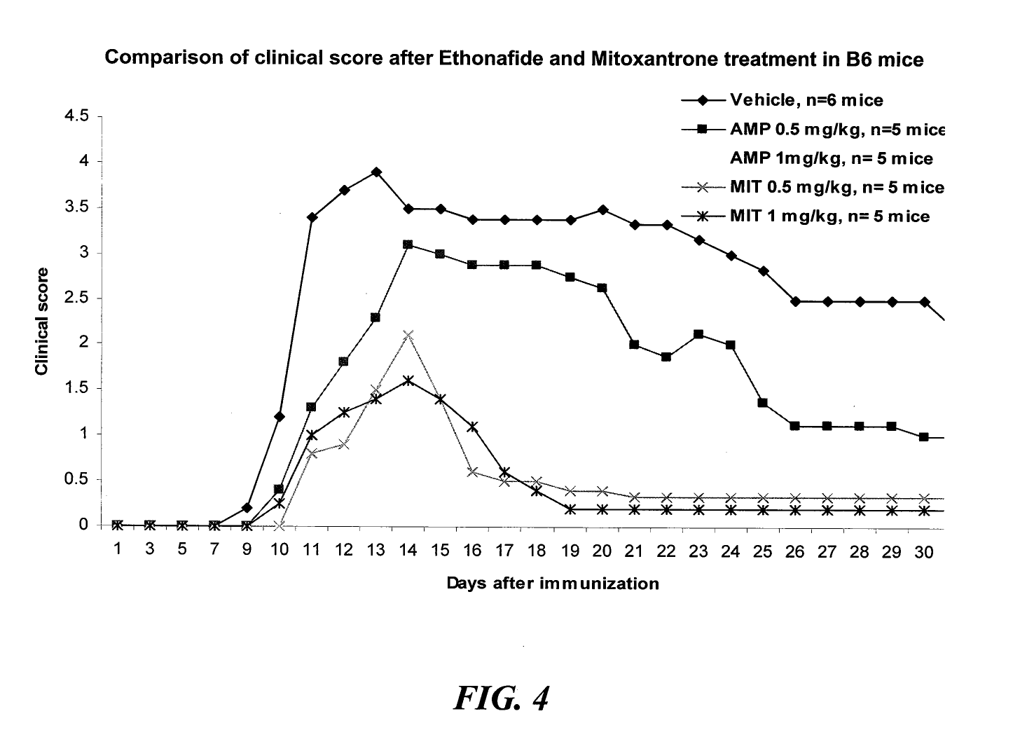

With reference now to FIG. 4, five groups of mice (each group containing 5 or 6 individuals), immunized for acute monophasic EAE as described hereinbefore, were treated with either ethonafide (AMP) or mitoxantrone at a dose of 0.5 mg/kg or 1.0 mg/kg beginning on day 11 following immunization, for seven days, or were left untreated to be used as a control. Each drug was administered intraperitoneally in phosphate buffered saline (untreated mice received i.p. administrations of the PBS vehicle). The mice were observed daily for clinical signs of disease and were scored as indicated hereinbefore on a clinical scale from 0 to 5. The incidence of disease in the immunized mice, the day of disease onset, the maximum clinical score, and the mean clinical score over a 32 day observation period were determined, and the results are shown in Table 3.

TABLE 3
Acute Monophasic EAE - Drug Administration beginning on day 11 post Immunization

| Drug - Dose (mg/kg) | Incidence | | Day of Onset | Max. Clinical Score | Mean Clinical Score* |
|---|---|---|---|---|---|
| PBS Vehicle | 100% | (6/6) | 10.4 ± 0.17 | 4.2 ± 0.16 | 2.87 ± 0.32 |
| Ethonafide - 0.5 | 100% | (5/5) | 11.8 ± 0.72 | 3.9 ± 0.26 | 1.86 ± 0.35 |
| Mitoxantrone - 0.5 | 100% | (5/5) | 12.2 ± 0.68 | 2.4 ± 0.36 | 0.59 ± 1.67 |
| Ethonafide - 1.0 | 60% | (3/5) | 12.3 ± 0.55 | 3.7 ± 0.22 | 1.17 ± 0.24 |
| Mitoxantrone - 1.0 | 60% | (3/5) | 13.3 ± 0.55 | 2.8 ± 0.36 | 0.51 ± 0.21 |

*calculated for mice that had evidence of disease.

As illustrated in FIG. 4, and in Table 3, the administration of ethonafide demonstrated a dose dependent effect in the treatment of clinical symptoms induced in the EAE mouse model.

As will be appreciated, the foregoing examples are offered only to illustrate, but not to limit, the claimed invention.

### III. Pharmaceutical Compositions of 1,2-dihydro-3H-dibenz(deh)isoquinoline-1,3-diones

Pharmaceutical compositions in accordance with aspects of the present invention may comprise a compound of Formula (I) or (II), wherein R₁, R₂, R₃, R₄, R₅, R₆, and R₇ are as described hereinbefore, pharmaceutically acceptable salts, prodrugs, or mixtures thereof, and optionally pharmaceutically acceptable additives and excipients.

A pharmaceutical formulation of the present invention can be micronized or powdered so that it is more easily dispersed and solubilized by the body. Processes for grinding or pulverizing drugs are well known in the art, for example, by using a hammer mill or similar milling device.

Dosage forms (compositions) suitable for internal administration contain from about 1.0 milligram to about 5000 milligrams of active ingredient per unit. In these pharmaceutical formulations, the active ingredient (e.g. azonafide compound, or other 1,2-dihydro-3H-dibenz(deh)isoquinoline-1,3-dione derivative compound) may be present in an amount of about 0.5 to about 95% by weight based on the total weight of the composition. Another convention for denoting the dosage form is in mg per meter squared (mg/m²) of body surface area (BSA). Typically, an adult will have approximately 1.75 m² of BSA. Based on the body weight of the patient, the dosage may be administered in one or more doses several times per day or per week. Multiple dosage units may be required to achieve a therapeutically effective amount. For example, if the dosage form is 1000 mg, and the patient weighs 40 kg, one tablet or capsule will provide a dose of 25 mg per kg for that patient. It will provide a dose of only 12.5 mg/kg for an 80 kg patient.

By way of general guidance, for humans a dosage of as little as about 0.1 milligram (mg) per kilogram (kg) of body weight, and up to about 100 mg per kg of body weight, is suitable as a therapeutically effective dose. Preferably, from about 0.1 mg/kg to about 50 mg/kg of body weight is used. Other preferred doses range between 5 mg/kg to about 25 mg/kg of body weight. However, a dosage of between about 1 mg/kg of body weight to about 35 mg/kg of body weight is also suitable.

Intravenously, the most preferred rates of administration can range from about 0.1 to about 10 mg/kg/minute during a constant rate infusion. A pharmaceutical formulation of the present invention can be administered in a single daily dose, or the total daily dosage may be administered in divided doses of two, three, or four times daily. A 1,2-dihydro-3H-dibenz(deh)isoquinoline-1,3-dione derivative compound is generally given in one or more doses on a daily basis or from one to three times a week.

A pharmaceutical formulation of the present invention is administered by any conventional means available for use in conjunction with pharmaceuticals, either as individual therapeutic agents or in combination with other therapeutic agents.

The amount and identity of the 1,2-dihydro-3H-dibenz(deh)isoquinoline-1,3-dione derivative compound can vary according to patient response and physiology, type and severity of side effects, the clinical pattern of disease being treated, the preferred dosing regimen, patient prognosis or other such factors.

It is contemplated that the compounds and compositions useful in accordance with aspects of the present invention may be used as part of a combination therapy with immune modulating agents such as interferon β and glatiramer acetate. Where the 1,2-dihydro-3H-dibenz(deh)isoquinoline-1,3-dione derivative compound is administered as part of a combination therapy, the respective doses and the dosing regimen of the 1,2-dihydro-3H-dibenz(deh)isoquinoline-1,3-dione derivative compound and the second therapeutic agent can vary. In one embodiment, the combination agent (e.g., glatiramer acetate) can be administered during periods between doses of the 1,2-dihydro-3H-dibenz(deh)isoquinoline-1,3-dione derivative compound. The exact regimen will depend on the clinical pattern of disease being treated, the severity of the symptoms of disease, and the patient's response to the treatment.

A 1,2-dihydro-3H-dibenz(deh)isoquinoline-1,3-dione derivative compound can be administered in accordance with the teachings of the present invention in oral dosage forms such as tablets, capsules, pills, powders, granules, elixirs, tinctures, suspensions, syrups, and emulsions. A composition comprising a 1,2-dihydro-3H-dibenz(deh)isoquinoline-1,3-dione derivative compound can also be administered in intravenous (bolus or infusion), intraperitoneal, subcutaneous, or intramuscular form, all using dosage forms well known to those of ordinary skill in the pharmaceutical arts.

A 1,2-dihydro-3H-dibenz(deh)isoquinoline-1,3-dione derivative compound is typically administered in accordance with the present invention in admixture with suitable pharmaceutical diluents, extenders, excipients, or carriers (collectively referred to herein as a pharmaceutically acceptable carrier or carrier materials) suitably selected with respect to the intended form of administration and as consistent with conventional pharmaceutical practices. The unit will be in a form suitable for oral, rectal, topical, intravenous injection or parenteral administration.

The pharmaceutical formulations can be administered alone or can be mixed with a pharmaceutically acceptable carrier. This carrier can be a solid or liquid, and the type of carrier is generally chosen based on the type of administration being used.

Specific examples of pharmaceutically acceptable carriers and excipients that can be used to formulate oral dosage forms of the present invention are well known to one skilled in the art. See, for example, U.S. Pat. No. 3,903,297, which is incorporated herein by reference in its entirety for all purposes. Techniques and compositions for making dosage forms useful in the present invention are also well known to one skilled in the art. See, for example, 7 Modern Pharmaceutics, Chapters 9 and 10 (Banker & Rhodes, Eds., 1979); Pharmaceutical Dosage Forms: Tablets (Lieberman et al., 1981); Ansel, Introduction to Pharmaceutical Dosage Forms 2^{nd} Ed. (1976); Remington's Pharmaceutical Sciences, 17^{th} ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), all of which are incorporated herein by reference in their entirety for all purposes.

Tablets can contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Pharmaceutical formulations can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Pharmaceutical formulations can also be coupled to soluble polymers as targetable drug carriers or as a prodrug. Suitable soluble polymers include polyvinylpyrrolidone, pyran copolymer, polyhydroxylpropylmethacrylamide-phenol, polyhydroxyethylasparta-midephenol, and polyethyleneoxide-polylysine substituted with palmitoyl residues. Furthermore, a 1,2-dihydro-3H-dibenz(deh)isoquinoline-1,3-dione derivative compound in accordance with aspects of the present invention can be coupled to a class of biodegradable polymers useful in achieving controlled release of a drug, for example, polylacetic acid, polyglycolic acid, copolymers of polylacetic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules can contain the active ingredient and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

Pharmaceutical formulations can also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms can also include minerals and other materials to make them compatible with the type of injection or delivery system chosen.

Useful pharmaceutical dosage forms for administration of an azonafide or other 1,2-dihydro-3H-dibenz(deh)isoquinoline-1,3-dione derivative compound are illustrated as follows:

A. Capsules

A large number of unit capsules may be prepared by filling standard two-piece hard gelatin capsules each with powdered active ingredient (e.g. 10 to 500 milligrams) and one or more of the following: lactose (e.g. 5 to 150 milligrams), cellulose (e.g. 5 to 50 milligrams), and magnesium stearate (e.g. 6 milligrams).

B. Soft Gelatin Capsules

A mixture of active ingredient in a digestible oil such as soybean oil, cottonseed oil or olive oil may be prepared and injected by means of a positive displacement pump into gelatin to form soft gelatin capsules containing the active ingredient (e.g. 100-500 milligrams). The capsules may then be washed and dried.

C. Tablets

A large number of tablets may be prepared by conventional procedures so that the appropriate dosage unit of active ingredient (e.g. 100-500 milligrams) is included along with one or more of the following: colloidal silicon dioxide (e.g. 0.2 milligrams), magnesium stearate (e.g. 5 milligrams), microcrystalline cellulose (e.g. 50-275 milligrams), starch (e.g. 11 milligrams) and lactose (e.g. 98.8 milligrams). Appropriate coatings may be applied to increase palatability or delay absorption.

D. Injectable Solution

A parenteral composition suitable for administration by injection may be prepared by stirring the appropriate amount of active ingredient (e.g. 1.5% by weight) in 10% by volume propylene glycol and water. The solution may be made isotonic with sodium chloride and sterilized.

E. Suspension

An aqueous suspension may prepared for oral administration so that each 5 ml contain the appropriate amount of finely divided active ingredient (e.g. 100 mg) and one or more of the following: sodium carboxymethyl cellulose (e.g. 200 mg), sodium benzoate (e.g. 5 mg), sorbitol solution (e.g. 1.0 g), and vanillin (e.g. 0.025 ml).

### IV. Pharmaceutical Kits

The present invention also includes pharmaceutical kits useful for the treatment of MS, which comprise one or more containers containing a pharmaceutical formulation comprising a therapeutically effective amount of a 1,2-dihydro-3H-dibenz(deh)isoquinoline-1,3-dione derivative compound. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit. It should be understood that although the specified materials and conditions are important in practicing the invention, unspecified materials and conditions are not excluded so long as they do not prevent the benefits of the invention from being realized.

Pharmaceutical carriers can be a solid or liquid and the type is generally chosen based on the type of administration being used. The active agents can be coadministered in the form of a tablet or capsule, liposome, as an agglomerated powder or in a liquid form. Examples of suitable solid carriers include lactose, sucrose, gelatin and agar. Capsules or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

### V. Methods of Treating Multiple Sclerosis Patients

Embodiments of the present invention provide methods for treating MS or a symptom thereof in a human patient in need of such treatment, and include administering to the patient a therapeutically effective amount of a 1,2-dihydro-3H-dibenz(deh)isoquinoline-1,3-dione derivative compound, or a pharmaceutical composition comprising a 1,2-dihydro-3H-dibenz(deh)isoquinoline-1,3-dione derivative compound in accordance with the present invention. It will be appreciated that MS symptoms or symptoms, as used herein, refers to clinical symptoms such as neurologic impairment and/or disability, as well as symptoms of disease such as neurological lesions, which may precede and/or accompany clinical symptoms.

Administration of a compound or pharmaceutical composition in accordance with the present invention can be accomplished by any suitable method, including oral, rectal, topical, parenteral, or intravenous administration. It is believed that parenteral treatment by intravenous, subcutaneous, or intramuscular application of a composition comprising a 1,2-dihydro-3H-dibenz(deh)isoquinoline-1,3-dione derivative compound formulated with an appropriate pharmaceutically acceptable carrier or diluent to facilitate application will be the preferred method of administering compounds in accordance with the present invention.

It is contemplated that the methods of the present invention, wherein a compound or pharmaceutical composition or formulation, as hereinbefore described, is administered to a patient to treat or prevent a symptom of MS, may comprise one component of a combination therapy in which patients are treated with other immune modulating agents. In one embodiment, the combination therapy can include the administration of interferon βs (i.e., IFNβ-1a and IFNβ-1b) to patients between scheduled courses of administration of a 1,2-dihydro-3H-di benzisoquinoline-1,3-dione derivative compound in accordance with the present invention.

A. Measuring Response to Pharmaceutical Formulations

Methods of monitoring disease status and/or progression in MS patients comprise the use of, for example, clinical rating scales (e.g., the Kurtzke Expanded Disability Status scale), relapse rates, and magnetic resonance imaging (“MRI”) measurements of CNS lesions.

The Kurtzke Expanded Disability Status scale (“EDSS”) is commonly used to monitor the status and/or progression of MS patients' disease, and measures neurologic impairment and disability in order to arrive at an EDSS score. The EDSS is comprised of an ordinal scale ranging from 0.0 (normal exam) to 10.0 (death due to MS) in 0.5 increments with the EDSS score being a function of a clinician's measurement of eight areas of a patient's CNS, including pyramidal, brainstem, visual, cerebral, cerebellar, sensory, bowel and bladder, and other (including fatigue) to evaluate a patient's ability to move, the patient's memory, concentration, coordination, balance, and other relevant impairments to functional CNS systems. While the degree of impairment or disability can be different in any two patients having the same EDSS score, a score of 2.5 or less is indicative of no more than minimal neurologic impairment or disability, a score of no more than 5.5, but greater than 2.5 is indicative of moderate to severe impairment or disability, while a score of 6.0 or greater indicates increasingly severe disability.

Neurological rating scales, such as the EDSS, are particularly useful in measuring response to treatment methodologies because they provide a reliable method for comparing disease status and/or progression between patients receiving varying types or doses of treatment, and are familiar to those skilled in the art. Other clinical rating scales (e.g., the MS Functional Composite) can also be used to measure response to treatment in MS patients and will be familiar to those skilled in the art.

In addition to clinical rating scales, measurements obtained through the use of MRI, or another imaging technique, also play an important role in monitoring disease status and/or progression, and in measuring response to treatment. In many respects, MRI measurements are much more sensitive than clinical rating scales in detecting evidence of MS disease activity, although the relationship between MRI findings and the clinical condition of the patient is yet to be fully understood. The detection of neurological lesions (e.g., T₂-weighted lesions, gadolinium-enhancing lesions, and T₁ hypointense lesions) provides an objective measure of disease progression, or lack thereof, and can provide useful prognostic information regarding clinical disease status and/or evolution in MS patients, particularly when used in conjunction with clinical evaluations based on one or more ratings scales, and observations of relapse rates in patients.

B. Assessing Toxicity and Setting Dosing Regimens

Patients are assessed for toxicity with each course of therapy, typically looking at effects on liver function enzymes and renal function enzymes such as creatinine clearance or BUN, as well as effects on the bone marrow, typically suppression of granulocytes important for fighting infection and/or suppression of platelets important for hemostasis or stopping blood flow. For such myelosuppressive drugs, the nadir in these normal blood counts is reached between 1-3 weeks after therapy and recovery then ensues over the next 1-2 weeks. Based on the recovery of normal white blood counts, treatments may then be resumed.

Treatment schedules for the administration of compounds or pharmaceutical compositions in accordance with the present invention conventionally comprise cycles of treatment wherein a specified dose of a 1,2-dihydro-3H-dibenz(deh)isoquinoline-1,3-dione derivative compound is administered to a patient at defined intervals over the period of a cycle, and then repeated in each subsequent cycle. The period of a cycle may be defined in any suitable manner, and may comprise, for example, a twenty-one day cycle, a twenty-eight day cycle, or the like. Within the period of a cycle of treatment, the specified dose of a compound in accordance with the present invention can be administered to the patient at defined intervals, such as for example, for five consecutive days every other week (e.g., days 1-5 and 15-19 of a 28-day cycle), for five consecutive days every three weeks (e.g., days 1-5 of a 21-day cycle), once per week (e.g., days 1, 8 and 15 of a 21-day cycle), or the like.

C. Clinical Management of Patients

Following a treatment cycle in which a compound or pharmaceutical composition in accordance with the present invention is administered to MS patients at a pre-defined dose and schedule, patients will be evaluated for response to therapy by any one or more of the methods described hereinbefore (e.g., clinical rating scales and MRI scans), and for toxicity associated with the administration of the therapy.

The projected clinical objectives of administration of a therapeutically effective amount of a 1,2-dihydro-3H-dibenz(deh)isoquinoline-1,3-dione derivative compound or corresponding pharmaceutical composition in accordance with the methods of the present invention are: the prevention of progression to clinically symptomatic disease; the prevention of disability due to disease progression; reduction of the frequency, severity, and/or duration of relapse; relief of symptoms of MS, including clinical symptoms and lesion load; and promotion of repair processes to restore function previously diminished by the onset or progression of MS.

While the invention is described here in the context of a limited number of embodiments, and with reference to specific details and examples, the invention may be embodied in many forms without departing from the spirit of the essential characteristics of the invention. The exemplary and described embodiments, including what is described in the abstract of the disclosure, are therefore to be considered in all respects as illustrative and not restrictive. The scope of the invention is indicated by the appended claims rather than by the foregoing description, and all changes that come within the meaning and range of equivalency of the claims are intended to be embraced therein. All patents, patent applications, and other publications cited in this application are incorporated by reference in their entirety for all purposes.

What is claimed is:

1. A method of treating a multiple sclerosis patient, the method comprising administering a therapeutically effective amount of a compound of the formula: 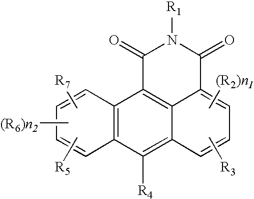
wherein,
R₁ is heteroalkyl, heterocycloalkyl, or heteroaryl;
R₂, R₄ and R₆ are independently hydrogen, halogen, nitro, amino, hydroxy, C₁-C₆ alkyl, C₁-C₆ heteroalkyl, heterocycloalkyl or aryl;
R₃, R₅, and R₇ are independently hydrogen or C₁-C₆ alkyl, or
R₃ and R₄ taken together with the carbon atoms to which they are attached form a phenyl ring, or
R₄ and R₅ taken together with the carbon atoms to which they are attached form a phenyl ring, or
R₅ and R₇ taken together with the carbon atoms to which they are attached form a phenyl ring; and
n₁ and n₂ are independently 0, 1 or 2;
or a pharmaceutically acceptable salt, a prodrug, or a mixture thereof.

2. The method of claim 1, wherein the amount is sufficient to prevent disability caused by disease progression.

3. The method of claim 1, wherein the amount is sufficient to reduce the frequency, severity, or duration of a relapse as compared to an untreated patient.

4. The method of claim 1, wherein the amount is sufficient to relieve one or more symptoms of multiple sclerosis.

5. The method of claim 1, wherein R₁ is —(CH₂)₂N(CH₃)₂.

6. The method of claim 5, wherein R₃, R₄, R₅, R₆, and R₇ are hydrogen, and R₂ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

7. The method of claim 5, wherein R₂, R₃, R₅, R₆, and R₇ are hydrogen, and R₄ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

8. The method of claim 5, wherein R₂, R₃, R₄, R₅, and R₇ are hydrogen, and R₆ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

9. The method of claim 6, wherein R₂ is attached to the dibenzisoquinoline ring structure at position 6 and is selected from —O(CH₂)₂N(CH₃)₂, —SCH₃, —SCH₂CH₃, —N(CH₃)₂, and —NH(CH₂)₂N(CH₃)₂, and wherein n₁ is 1.

10. The method of claim 6, wherein R₂ is attached to the dibenzisoquinoline ring structure at position 6 and is —OCH₂CH₃, and wherein n₁ is 1.

11. The method of claim 1, wherein R₁ is selected from —(CH₂)₂N(CH₃)₂, —(CH₂)₂NHCH₃, —(CH₂)₃N(CH₃)₂, —(CH₂)₂NH(CH₂)₂OH, —(CH₂)₃N(CH₂CH₂OH)₂, —(CH₂)₂N(CH₂)₄, —(CH₂)₂N(CH₂)₅, —(CH₂)₂-(1-methyl-2-pyrrolidinyl), —CH₂-(1-ethyl-2-pyrrolidinyl), -(1-ethyl-3-piperidinyl), —(CH₂)₂—N-morpholinyl, —(CH₂)₂—N-piperazinyl, —(CH₂)₂-2-pyridyl, —CH₂-2-pyridyl, —CH₂-3-pyridyl, -(3-pyridyl), and -(p-N(CH₃)₂)—C₆H₅), and wherein R₂, R₃, R₄, R₅, R₆, and R₇ are hydrogen.

12. The method of claim 1, wherein the compound, pharmaceutically acceptable salt, or prodrug comprises a component of a pharmaceutical composition suitable for administration to the patient.

13. A method of treating a patient having a neurological lesion associated with multiple sclerosis, the method comprising administering a therapeutically effective amount of a compound of the formula: 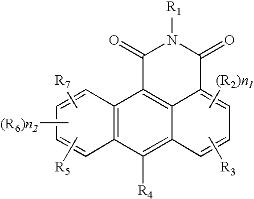
wherein,
R₁ is heteroalkyl, heterocycloalkyl, or heteroaryl;
R₂, R₄ and R₆ are independently hydrogen, halogen, nitro, amino, hydroxy, C₁-C₆ alkyl, C₁-C₆ heteroalkyl, heterocycloalkyl or aryl;
R₃, R₅, and R₇ are independently hydrogen or C₁-C₆ alkyl, or
R₃ and R₄ taken together with the carbon atoms to which they are attached form a phenyl ring, or
R₄ and R₅ taken together with the carbon atoms to which they are attached form a phenyl ring, or
R₅ and R₇ taken together with the carbon atoms to which they are attached form a phenyl ring; and
n₁ and n₂ are independently 0, 1 or 2;
or a pharmaceutically acceptable salt, a prodrug, or a mixture thereof.

14. The method of claim 13, wherein the patient exhibits no neurologic impairment or disability as measured by a clinical rating scale.

15. The method of claim 14, wherein the amount is sufficient to prevent progression to clinically symptomatic multiple sclerosis as measured by the clinical rating scale.

16. The method of claim 13, wherein the patient exhibits no more than minimal neurologic impairment or disability as measured by a clinical rating scale.

17. The method of claim 16, wherein the amount is sufficient to prevent disease progression as measured by the clinical rating scale.

18. The method of claim 13, wherein R₁ is —(CH₂)₂N(CH₃)₂.

19. The method of claim 18, wherein R₃, R₄, R₅, R₆, and R₇ are hydrogen, and R₂ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

20. The method of claim 18, wherein R₂, R₃, R₅, R₆, and R₇ are hydrogen, and R₄ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

21. The method of claim 18, wherein R₂, R₃, R₄, R₅, and R₇ are hydrogen, and R₆ is selected from —CH₃, —CH₂—CH₃, —CH₂—CH₂—CH₃, —F, —Cl, —I, —Br, —OH, —OCH₃, —OCH₂—CH₃, —OCH₂—CH₂—CH₃, —OCH₂—CH₂—N(CH₃)₂, —OCH₂—CH₂—N(CH₂CH₃)₂, —NH₂, —N(CH₃)₂, —N(CH₂CH₃)₂, —NH—CH₂—CH₂—OH, —NHCOCH₃, —NHCOC(CH₃)₃, —NH—CH₂—CH₂—N(CH₃)₂, —NH—CH₂—CH₂—N(CH₂CH₃)₂, —NO₂, —CN, —SCH₃, —SCH₂CH₃, —SCH₂—CH₂—CH₃, and —SO₂CH₃.

22. The method of claim 19, wherein R₂ is attached to the dibenzisoquinoline ring structure at position 6 and is selected from —O(CH₂)₂N(CH₃)₂, —SCH₃, —SCH₂CH₃, —N(CH₃)₂, and —NH(CH₂)₂N(CH₃)₂, and wherein n₁ is 1.

23. The method of claim 19, wherein R₂ is attached to the dibenzisoquinoline ring structure at position 6 and is —OCH₂CH₃, and wherein n1 is 1.

24. The method of claim 13, wherein R₁ is selected from —(CH₂)₂N(CH₃)₂, —(CH₂)₂NHCH₃, —(CH₂)₃N(CH₃)₂, —(CH₂)₂NH(CH₂)₂OH, —(CH₂)₃N(CH₂CH₂OH)₂, —(CH₂)₂N(CH₂)₄, —(CH₂)₂N(CH₂)₅, —(CH₂)₂-(1-methyl-2-pyrrolidinyl), —CH₂-(1-ethyl-2-pyrrolidinyl), -(1-ethyl-3-piperidinyl), —(CH₂)₂—N-morpholinyl, —(CH₂)₂—N-piperazinyl, —(CH₂)₂-2-pyridyl, —CH₂)₂-pyridyl, —CH₂-3-pyridyl, -(3-pyridyl), and -(p-N(CH₃)₂)—C₆H₅), and wherein R₂, R₃, R₄, R₅, R₆, and R₇ are hydrogen.

25. The method of claim 13, wherein the compound, pharmaceutically acceptable salt, or prodrug comprises a component of a pharmaceutical composition suitable for administration to the patient.
